(19) United States
(12) Patent Application Publication
(10) Pub. No.: US 2026/0225075 A1
(43) Pub. Date: Aug. 6, 2026

(54) SUPPORTED RU CATALYSTS HIGHLY EFFICIENT FOR CARBON DIOXIDE METHANATION, METHODS OF PREPARATION AND USES THEREOF

(71) Applicant: GAZNAT SA, LAUSANNE (CH)

(72) Inventors: LIPING ZHONG, SION (CH); ANDREAS ZÜTTEL, SION (CH)

(21) Appl. No.: 19/151,670

(22) PCT Filed: Jan. 29, 2024
(86) PCT No.: PCT/EP2024/052020

§ 371 Date: Jul. 29, 2025

(30) Foreign Application Priority Data
Jan. 30, 2023 (EP) 23154069.1

(51) Int. Cl. B01J 21/04; B01J 23/46; B01J 35/51; B01J 35/55; B01J 35/61; B01J 35/63; B01J 37/02; B01J 37/08; C07C 1/04; C07C 1/12

(52) U.S. Cl. CPC B01J 21/04; B01J 23/462; B01J 35/51; B01J 35/55; B01J 35/615; B01J 35/633; B01J 37/0207; B01J 37/0236; B01J 37/08; C07C 1/0435; C07C 1/12; C07C 2521/04; C07C 2523/46

(57) ABSTRACT

The invention relates to a method for the preparation of supported Ru nanoparticles material, in particular Ru/Al₂O₃ and heterogenous catalyst comprising Ru metal particles and Al₂O₃ useful in CO₂ methanation.

### FIELD OF THE INVENTION

The present invention pertains generally to the field of supported Ru catalysts for use in CO₂ methanation. The invention more specifically relates to γ-Al₂O₃-supported Ru nanoparticles catalysts and methods of preparation thereof and uses of those.

### BACKGROUND OF THE INVENTION

In order to overcome the shortage of fossil fuels in next generations, the development of renewable energy is crucial. Hydrocarbons synthesized from CO₂ captured from air and hydrogen obtained from water via electrolysis is considered not only as renewable chemical energy carrier but also a process to close the carbon cycle (Moioli et al., 2019, Renewable and Sustainable Energy Reviews 107, 497-506; Gotz et al., 2016, Renewable Power-to-Gas: A technological and economic review. Renewable Energy 85, 1371-1390).

Current technology allows the conversion of CO₂ and hydrogen to methane with high selectivity via Sabatier reaction. The Sabatier reaction was first described in 1910 by French chemist Paul Sabatier and has important applications, for example as a power-to-gas process. In this process, the methane produced from renewable H₂ and CO₂ can be injected into the existing gas network or stored for transportation or heating application (Lewandowska-Bernat et al., 2018, Applied Energy, 228, 57-67; Falbo et al., 2018, Applied Catalysis B: Environmental 225, 354-363; Schiebahn et al., 2015, International Journal of Hydrogen Energy 40, 4285-4294). During last decades, considerable efforts have been devoted to the development of adequate catalyst and reactors for the Sabatier reaction due to the arising attention given to CO₂ emissions.

According to previous research, the synthesis of CH₄ by CO₂ hydrogenation can be achieved by utilizing metal catalysts such as Ru, Ni, Co, Pt, Rh etc., respectively (Frontera et al., 2017, Catalysts vol. 7, Ashok, et al., 2020, Catalysis Today 356, 471-489; Mutschler et al., 2019, Journal of Catalysis 375, 193-201).

Among them, Ru-based catalysts have shown a superior activity of high selectivity and long stability. Since CO₂ hydrogenation to form methane is an exothermic process, in order to guarantee high conversion of CO₂ and 100% of CH₄ selectivity, the low temperature activity of catalyst is fundamental. Even though a high number of publications have shown that nickel or cobalt catalysts can obtain a comparable reactivity of ruthenium under certain condition, the sintering of metal particles as well as high activation temperature is always a challenge for industrialization of power-to-gas processes with using nickel-based catalyst (Frontera et al., 2017, supra; Ashok, et al., 2020, supra). Moreover, a commercial Ru_{0.5wt. %}/Al₂O₃ catalyst (Sigma-Aldrich #206199) has shown a 99% of CO₂ conversion and 99% of CH₄ at temperatures between 250° C. to 300° C. without recycling the reactants (Gallandat et al., 2018, Sustainable Energy and Fuels 2, 1101-1110). To reduce the catalyst cost caused by the high price of ruthenium, it is necessary to synthesize a ruthenium catalyst with higher mass specific activity and reduce the cost of manufacturing by simplifying the manufacturing process.

Ruthenium supported catalyst is conventionally synthesized via the impregnation and precipitation methods, respectively (Peng et al., 2016, ChemCatChem, 8, 139-141; U.S. Pat. No. 4,049,584; Zeng et al., 1997, Applied Catalysis B: Environmental, vol. 13; Lin et al., 2019, ACS Catalysis, 9, 1635-1644; Kowalczyk et al., 2008, Applied Catalysis A: General 342, 35-39; Liang et al., 2012, International Journal of Hydrogen Energy 37, 17921-17927). In the conventional impregnation process, the factory support material (e.g., Al₂O₃ or TiO₂) is impregnated with an aqueous solution of ruthenium component such as RuCl₃·xH₂O, RuN₄O₁.xH₂O and ruthenium (III) acetate, etc. This method has been widely used in industry due to its simplicity and high yield of metal component (Munnik et al., 2015, Chemical Reviews, vol. 115, 6687-6718; Adrian et al., 2020, CATALYSIS TODAY, 356, 419-432; Baddour F, The Engineering of Catalyst Scale Up, BETO peer review 2021, p. 20-21). In most of patents and articles which have used the impregnation method for the synthesis of Ru/Al₂O₃ catalysts, the size and distribution of Ru metal particles on the support significantly varies with the loading of Ru (Bobadilla et al., 2019, Chemical Engineering Journal, 357, 248-257). Even though this method is simple, it has several drawbacks, the most important being the uneven distribution of metal particles in size and shape (Okal et al., 2007, Applied Catalysis A: General, 319, 202-209). For instance, in EP1048347, the Ru/Al₂O₃ catalyst with a Ru weight percentage from 0.5-5% comprises Ru particles with a wide size distribution between 0.5 nm-100 nm. Yan et al., 2018, Journal of Catalysis, 367, 194-205, use a water-insoluble ruthenium precursor, the ruthenium acetylacetonate, along with ethanol as a solvent for the precursor solution and is preferred over ruthenium chloride since ruthenium (III) acetylacetonate for synthesizing Ru/Al₂O₃ catalysts because it has a templating effect which helps the dispersion of Ru resulting in better reactivity for CO₂ methanation (Renda et al, 2020, Applied Energy, 279, 115767). However, this alternative precursor and the solvent needed result in high costs due to raw material pricing. Further, the STEM images of the 3% Ru/Al₂O₃ sample reveal that the measured size of Ru particles (depicted as bright dots) based on the scale-bar averages is above 4 nm and the Ru/Al₂O₃ catalyst prepared with a 1% Ru loading, achieved only around 5% CO₂ conversion at 300° C. Moreover, if RuCl₃.xH₂O is used as precursor, the contamination of Cl⁻ on the surface of the metal cannot be avoided (Gates et al., 1995, Chem. Rev, vol. 95, 511-522). Another source of metal contamination in such systems may be the alumina support dissolved in highly acidic solutions used for impregnation (Munnik et al., 2015, supra; Gates et al., 1995, supra; Maki-Arvela et al., 2013, Applied Catalysis A: General, 451, 251-281). Besides, it was reported that the size of Ru increased with the Ru weight percentage and affects dramatically the CH₄ selectivity of CO₂ methanation (Kwak et al., 2013, ACS Catalysis, 3, 2449-2455). An alternative method to obtain supported Ru/Al₂O₃ is by reduction of RuCl₃ in ethylene glycol (Chen et al., 2008, Materials Letters 62, 1018-1021; U.S. Pat. No. 9,499,402B2). Usually, supported Ru/Al₂O₃ catalysts are prepared by impregnating the Al₂O₃ support into ruthenium solution and then reducing it in H₂ at high temperature or using glycol reduction methods to synthesize Ru colloid and then deposit it onto the Al₂O₃ support. In this method, the distribution and dispersion of Ru particles strongly depends on the surface properties of the different Al₂O₃ products and the Ru size is affected by various factor such as ruthenium loading, drying temperature, calcination temperature and gas environment. In this process, almost all of the ruthenium ions could be reduced to metallic state by the polyol agent and chlorine ions could be easily removed by the solution. However, the KNO₃/NaNO₃ used in this process always lead to impurities of K or Na in the final Ru/Al₂O₃ product. The existence of K and Na is reported to have negative effects on the CO₂ methanation reactivity towards not only the CO₂ conversion but also to the selectivity of methane (Cimino et al., 2020, Journal of CO₂ Utilization, 37, 195-203). However, as it is known, the properties of Ru nanoparticles in terms of size, dispersion and its interaction with the support significantly influences their catalytic performance (both activity and stability) (Xu et al., 2016, Journal of Catalysis 333, 227-237).

Although several patents or articles have reported the synthesis of Ru/Al₂O₃ supported catalyst via the impregnation method, none of them has claimed a process to get highly dispersed Ru metal particles with a narrow profile of Ru size distribution.

Therefore, there is need for developing cost-effective methods of preparation of Ru/Al₂O₃ catalysts on an industrial scale to obtain a product with high activity and selectivity as well as long-term stability under the harsh conditions of CO₂ methanation processes.

### SUMMARY OF THE INVENTION

A general object of this invention is to provide supported Ru particles as a highly active, selective as well as durable catalyst for CO₂ methanation and a cost-effective process for the preparation of the same.

One of the specific objects of this invention is to provide a process for the preparation of supported Ru particles, in particular Ru/Al₂O₃ material, which are useful as catalyst for CO₂ methanation.

It is advantageous to provide a facile and scalable method for the preparation of supported Ru particles.

It is advantageous to provide a method for the preparation of supported Ru particles allowing the control of the size of Ru particles.

It is advantageous to provide a method for the preparation of supported Ru particles adapted to different porous Al₂O₃ materials.

It is advantageous to provide a process for preparing a catalyst with highly dispersed ruthenium metal particles with small and stable particle size.

It is advantageous to provide a facile and scalable method for the preparation of supported Ru particles using incipient wetness impregnation.

An object of this invention is to provide a heterogenous catalyst comprising Ru metal particles supported by Al₂O₃ useful for CO₂ methanation.

It is advantageous to provide Ru/Al₂O₃ material with a narrow profile of Ru particle size distribution within the Al₂O₃.

It is advantageous to provide Ru/Al₂O₃ material with a small size of Ru particles with improved mass specific reactivity in CO₂ methanation processes, thereby improving the economic efficiency of ruthenium supported catalysts.

It is advantageous to provide Ru/Al₂O₃ material presenting a CO₂ conversion efficiency of more than 40% at a temperature lower than 300° C. for a cost-effective amount of Ru loading (typically a Ru loading lower than 1%).

It is advantageous to provide Ru/Al₂O₃ particles presenting strong resistance to sintering or agglomeration under high temperature conditions.

Objects of this invention have been achieved by providing a method of preparation according to claim 1, a and uses thereof according to claim 15.

Disclosed herein is a method for the preparation of Al₂O₃ supported Ru particles comprising the steps of:
a) Providing a porous Al₂O₃ support material which has been pre-treated in an ammonia solution;
b) Providing a low acidic ruthenium precursor solution, in particular a ruthenium precursor solution comprising soluble ruthenium salts in aqueous solution, wherein the Ru mass content in the Ru precursor solution is from about 0.1 g/L to about 5 g/L;
c) Impregnating the surface of the said pre-treated Al₂O₃ support material with the ruthenium precursor solution by incipient wetness impregnation;
d) Drying the obtained impregnated Ru/Al₂O₃ material;
e) Subjecting the dried impregnated Ru/Al₂O₃ material to temperature treatment under an H₂ atmosphere to reduce the ruthenium precursor into Ru metal phase;
f) Contacting the obtained Ru/Al₂O₃ material to an ammonia solution;
g) Drying the obtained ammonia-treated Ru/Al₂O₃ material.

Also disclosed herein is Ru metal particles supported by Al₂O₃ obtainable by a process according to the invention.

Also disclosed herein is a heterogenous catalyst comprising Ru metal particles and Al₂O₃, wherein said catalyst has a mass loading of Ru from 0.1% wt to 2.5% wt and a size of Ru particles less than 2 nm.

Also, disclosed herein is a use of Ru metal particles supported by Al₂O₃ as a catalyst in CO₂ methanation.

Other features and advantages of the invention will be apparent from the claims, detailed description, and figures.

### BRIEF DESCRIPTION OF THE DRAWINGS

FIG. 1 is a schematic representation of an illustration of the steps of a method of preparation of Ru metal particles supported by Al₂O₃ of the invention.

FIG. 2 shows STEM images of Examples of the invention and commercial Ru/Al₂O₃ sample as described in Example 7.

FIG. 3 represents the CH₄ formation rate during CO₂ hydrogenation reaction using Ru/Al₂O₃ catalysts of the invention compared to comparative commercial catalyst (CS) and comparative Example 1 (C) as described in measured under same conditions of 285° C., 1 bar, GHSV=6 L/g/h.

FIG. 4 represents the percentage of CO₂ conversion versus time on stream (T) as described in Example 8 under an accelerated reaction condition: 10 bar, T=310° C., GHSV=6 L/g/h.

FIG. 5 shows the behavior of the Ru particle size of samples of a catalyst of the invention (Ru0.5_{wt}%/Al₂O₃) calcined in 5% H₂ in N₂ balance at different temperatures as illustrated by STEM images (A) and calculated size distribution (B) for a sample with a calcination at 400° C. (left) and 800° C. (right), and the representation of the evolution of the average particle size versus temperature (C) as described in Example 1 and 6.

FIG. 6 shows the characterization of a catalyst sample produced in a scale of 5 kg by the process of the invention in terms of Ru particle size distribution (A) and CO₂ conversion activity (1) compared with the same catalyst synthesized in laboratory conditions (2) (B) as described in Example 9. E represents the thermodynamic equilibrium.

FIG. 7 shows the Ru Size of samples prepared by different-steps impregnation of RuCl₃.xH₂O solution with same concentration of 3 mg/ml Ru as described in Example 7.

FIG. 8 shows the CO₂ conversion of Ru 0.5 wt %/Al₂O₃ samples synthesized with RuCl₃.xH₂O but pretreated with water, ammonia respectively as described in Example 7.

FIG. 9 shows CO₂ conversion (%) of Ru/Al₂O₃ catalysts with 0.5 wt % loading of ruthenium synthesized with non-pretreated Al₂O₃ (not the invention) and ammonia solution (3M for 3 h) pretreated Al₂O₃ as described in Example 7.

### DETAILED DESCRIPTION OF EMBODIMENTS OF THE INVENTION

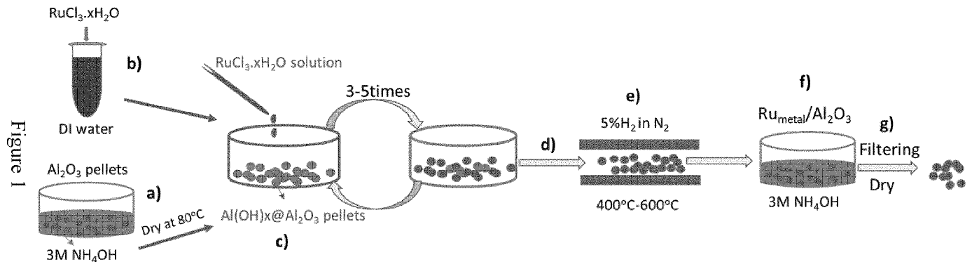

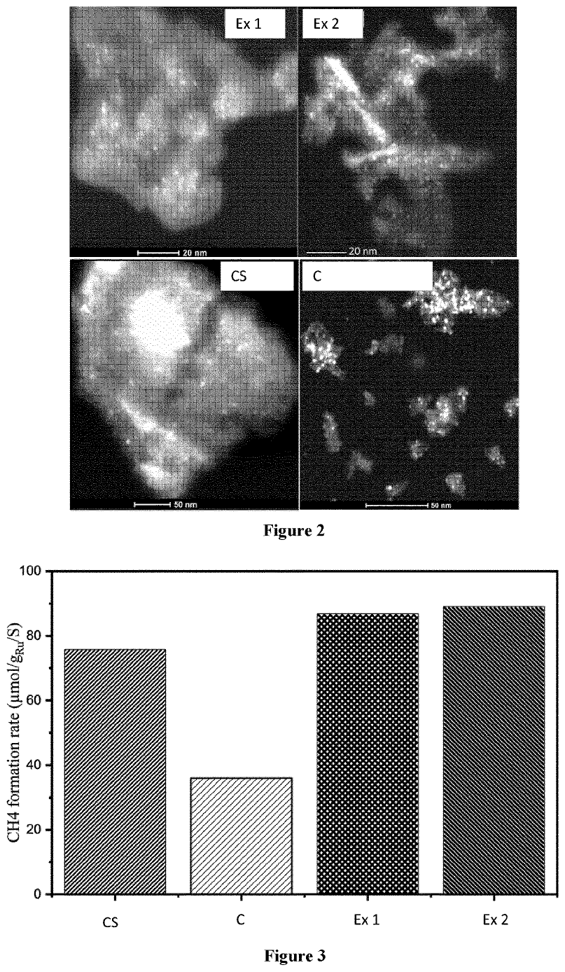

Referring to the figures, in particular first to FIG. 1, is provided an illustration of a method for the preparation of supported Ru particles.

More specifically, the steps of the embodiment illustrated in FIG. 1 comprise:
a) Providing a porous Al₂O₃ support material which has been pre-treated in an ammonia solution;
b) Providing a ruthenium precursor solution comprising soluble ruthenium salts such as RuCl₃ or ruthenium nitrosyl nitrate salt in aqueous solution, wherein the Ru mass content in the Ru precursor solution is between 0.1 g/L and 5 g/L;
c) Impregnating the surface of the said pre-treated Al₂O₃ support material with the ruthenium precursor solution by incipient wetness impregnation;
d) Drying the obtained impregnated Ru/Al₂O₃ material;
e) Subjecting the dried impregnated Ru/Al₂O₃ material to temperature treatment under H₂ atmosphere to reduce the ruthenium precursor into Ru metal phase;
f) Contacting the obtained Ru/Al₂O₃ material with an ammonia solution;
g) Drying the obtained ammonia-treated Ru/Al₂O₃ material.

According to a particular embodiment, the porous Al₂O₃ support material is provided in the form of cylindrically or spherically shaped Al₂O₃ pellets.

According to another particular embodiment, the porous Al₂O₃ support material is calcinated before subjecting to an ammonia solution to remove water and any adsorbents. Typically, calcination temperatures range from about 400 to about 800° C. (e.g. 500° C.).

According to a particular embodiment, the porous Al₂O₃ support material is pre-treated in an ammonia solution with a concentration from 1M to 3M for from about 3 to about 15 hours.

According to a particular embodiment, the porous Al₂O₃ support material has a specific surface area from or over 100 m²/g (e.g. from about 100 to about 1,000 m²/g, for example from 200 to about 500 m²/g) and a total pore volume from or over 0.4 cm³/g (e.g. from about 0.4 to about 2 cm³/g, typically from about 0.5 to about 1 cm³/g).

According to a particular aspect, this pre-treatment has the advantage of removing the possible impurities (e.g., chloride and sulfur) existing in Al₂O₃ material but also modifying the point of zero charge of Al₂O₃ surface which promotes a stable anchoring of Ru³⁺ ions on the Al₂O₃ surface.

According to a particular embodiment, the pre-treated Al₂O₃ support material is subjected to a drying step, for example at about 70-120° C. such as 70-90° C. (e.g. 80° C.) for about 2 to 15 hours prior the impregnation step.

According to further particular embodiment, the pre-treated Al₂O₃ support material is washed with deionized water before the drying steps.

According to another further particular embodiment, if the Al₂O₃ support material is not subjected to the impregnation step immediately after pre-treatment with the ammonia solution and washing/drying steps, it can be stored in a dry environment and air-tight environment at a temperature below 80° C., preferably below 20° C. for few hours but not more than about 2 days to avoid the transformation or decomposition of the Al(OH)x species.

According to a particular aspect, the concentration of Ru in the precursor solution is defined by the following equation (1):

$${C}_{Ru , solution} = \frac{{Ru}_{cata} \% \times 1.15 \times {m}_{cata}}{N \times {V}_{support} \times 1.2 \times {Ru}_{precursor} \%} \qquad equation ( 1 )$$

Wherein Ru_{cata}% is the target weight percentage of Ru metal content in the final catalyst; M_{cata} is the mass of final synthesized catalyst (g); N is the impregnation times, which should be a whole number to ensure the C_{Ru,solution} is less than 5 g/L.

According to a particular aspect, the Ru in the precursor solution is a Ruthenium Chloride hydrate (RuCl₃·xH₂O) solution.

According to another particular aspect, the Ru in the precursor solution is a Ruthenium nitrosyl nitrate solution.

According to a particular aspect, the Ru in the precursor solution is either freshly prepared shortly before the impregnation or has been prepared earlier (but not more than 48 hours) and has been stored at a temperature from 3 to less than 20° C., typically from about 3 to about 10° C. under an atmosphere protected from air exposure.

According to a particular aspect, the pre-treated Al₂O₃ support material is then impregnated with the ruthenium precursor solution such as a solution RuCl₃·xH₂O by incipient wetness impregnation.

According to a further particular embodiment, incipient wetness impregnation comprises at least one impregnation sequence, preferably at least 2 (typically 3 to about 5 sequences) of the following steps: i) wetting the pre-treated Al₂O₃ support material surface drop by drop with a volume of solution containing the ruthenium precursor corresponding to its pore volume until a slurry is finally formed when the whole pore volume of the support has been filled and ii) dry the impregnated Al₂O₃ support material, typically by heating in air or vacuum conditions to about 50° C. to 100° C. from about 30 min to few hours such as for example 2 hours.

According to a particular aspect, the number of impregnation sequences is defined by the support pore volume, the concentration of Ru in precursor aqueous solution and Ru loading content as set out in equation (1).

Conventionally, the concentration of Ru in the precursor solution used in impregnation step varies with the loading content of Ru on support and the total pore volume of Al₂O₃. The present invention has revealed that a constant Ru mass content in the Ru precursor aqueous solution between 0.1 g/L to about 5 g/L and preferably from about 2 g/L to 5 g/L (e.g. 3 g/L) will guarantee the small size and good dispersion of Ru nanoparticles.

According to a further particular embodiment, at each impregnation sequence, a volume of the Ru precursor solution 1.15 times the total volume of the pre-treated Al₂O₃ support will be dropped onto the pretreated Al₂O₃ support.

According to another further particular embodiment, at each impregnation sequence, after the Ru precursor aqueous solution is filled into the pores of Al₂O₃ via capillary force, the impregnated product is dried at temperatures between 20° C. to lower than 120° C., typically for about 30 min to about 2 h. The drying temperature should be strictly controlled under 120° C. to avoid Ru species to agglomerate during fast water evaporation.

According to another further particular embodiment, an evaporation rate of water less than 10 mg/m²/min is maintained during each drying step of the impregnation sequence. The evaporation rate can be determined by thermal gravimetric analysis measurements normalized to the surface area of catalyst support.

According to another further particular embodiment, the evaporation rate of water during each drying step of the impregnation sequence is between 0.2 mg/m²/min and 1.2 mg/m²/min (e.g. 0.5 mg/m²/min).

According to another further particular embodiment, at the end of the impregnation step, the obtained dried impregnated Al₂O₃ support material is subjected to a temperature treatment under reductive atmosphere such that the ruthenium precursor is reduced into Ru metal phase.

According to a further particular embodiment, the temperature treatment of the obtained dried impregnated Al₂O₃ support material is conducted in diluted H₂ atmosphere, typically at with a volume ratio from 1% to 20% in an inert gas of N₂, Argon or Helium.

According to a further particular embodiment, the temperature treatment of the obtained dried impregnated Al₂O₃ support material is conducted under N₂-diluted H₂ gas environment with a concentration of H₂ between 1%-20% mol/mol.

According to another further particular embodiment, the temperature treatment of the obtained dried impregnated Al₂O₃ support material is carried out at an annealing temperature from about 300° C. to about 800° C. (e.g. 400-700° C.).

According to another further particular embodiment, the temperature treatment is carried out with a heating rate not more than 10° C./min (typically from about 1° C./min to about 10° C./min) and dwell time of at least 2 hours (eg. From about 2 to 3 hours).

According to a further particular embodiment, the annealed Ru/Al₂O₃ material is contacted with an ammonia solution (e.g. 3M to 5M), for example by immersion in 3M of ammonia solution.

According to another further particular embodiment, the annealed Ru/Al₂O₃ material is contacted with an ammonia solution of 3M or higher, typically from 3M to 5M (e.g. 5.6 wt %) which increases the presence of OH groups lading the enhancement of Ru metal dispersion and modify the surface of Al₂O₃ material, thereby contributing to the creating more active sites for CO₂ conversion.

According to a further particular embodiment, the annealed Ru/Al₂O₃ material is immersed in an ammonia solution for about 1 to about 6 hours (e.g. from 1 to 5 hours).

According to a further particular embodiment, the ammonia-treated annealed Ru/Al₂O₃ material is dried before storage.

According to another particular embodiment, the ammonia-treated annealed Ru/Al₂O₃ material can be stored before use in a dry environment and air-tight environment at a temperature below 80° C.

According to another particular aspect, the obtained Ru/Al₂O₃ material is characterized by highly dispersed Ru particles, a narrow particle size distribution (typically from about 0.8 nm to about 1.5 nm), a long lifetime (typically more than 400 hours) and a high conversion of CO₂ to methane when used as a catalyst in CO₂ methanation processes, in particular via Sabatier reaction.

According to another particular aspect, the obtained Ru/Al₂O₃ material is characterized by a strong resistance to sintering or agglomeration under high temperature. In particular, Ru particles size keeps constant even after heating in H₂ up to 800° C.

According to another particular aspect, is provided a heterogenous catalyst comprising Ru metal particles supported by Al₂O₃, wherein said catalyst is obtainable by a process according to the invention.

According to another particular aspect, is provided a heterogenous catalyst comprising Ru metal particles and Al₂O₃, with a Ru mass content between 0.1% wt and 2.5% wt, while the size of Ru particles is less than 2 nm.

According to another particular aspect, is provided a heterogenous catalyst comprising Ru metal particles and Al₂O₃, with a Ru mass content between 0.1% wt and 2.5% wt, while the size of Ru particles is less than 2 nm and wherein presenting a CO₂ conversion efficiency of more than 40% at a temperature from about to 250° C. to about 300° C.

According to a particular aspect, the size of Ru metal particles within the catalyst of the invention ranges from about 0.5 to 1.5 nm such as from about 0.8 nm to about 1.5 nm.

According to a particular aspect, ruthenium particle size analysis utilizes Transmission Electron Microscopy (TEM), in particular specifically employing the STEM mode. To facilitate analysis, the Ru/Al₂O₃ catalyst can be dispersed in ethanol and deposited onto a carbon-coated copper TEM grid. The average diameter or size of the Ru particles is subsequently derived from a correlation based on this methodology (Karim et al., 2009, J. Am. Chem. Soc., 131, 34, 12230 12239).

$${d}_{avg} = {\sum }_{i} {n}_{i} {d}_{i} / {\sum }_{i} {n}_{i}$$

di the Ru particle diameter measured from STEM images scanning in the horizontal direction and ni is the number of particles. It is the most powerful and most straightforward method for determining the metal particle size distribution, especially in the case of supported noble metal catalysts (Mishra et al., 2013, Journal of Molecular Catalysis A Chemical 376:63-70; Berger et et al., Particle Size and Dispersion Measurements. Handbook of Heterogeneous Catalysis, 2, Wiley-VCH, pp. 738-765, 2008).

According to a particular aspect, the dispersion degree of Ru particles within the Al₂O₃ support is from about 0.17 atom to 0.3 atom per nm² (typically about 0.175 atom/nm²). The quantification of the dispersion degree can be assessed by standard methods such as described in Comas-Vives et al., 2016, Phys. Chem. Chem. Phys., 18, 1969-1979.

According to a particular aspect, the conversion rate of CO₂ to methane in presence of a catalyst of the invention is from about 80 μmo/g_{Ru}/s to about 90 μmo/g_{Ru}/s at 1 bar around 250° C. in a fixed bed micro reactor. The quantification of reactivity is based on equation (2) to equation (4) as indicated in the examples.

According to another particular aspect is provided the use of a Ru/Al₂O₃ material according to the invention for subjecting CO and/or CO₂ to methanation reaction with hydrogen.

According to another particular aspect is provided a method for producing methane, said method comprising bringing a catalyst according to the invention into contact with a mixed gas containing CO and/or CO₂ and a hydrogen gas at a temperature of at least 200° C.

Hence, the present invention provides a process based on the impregnation method which is able to produce a Ru/Al₂O₃ catalyst with a size of Ru particle less than 2 nm, and which has shown a better mass specific reactivity of Ru for methane yield as compared to commercial Ru_{0.5wt. %}/Al₂O₃ catalyst (Sigma-Aldrich #206199) and catalyst prepared via conventional methods.

The invention having been described, the following examples are presented by way of illustration, and not limitation.

### EXAMPLES

The method of the invention has been exemplified as follows with comparative examples shown below.

Ruthenium chloride hydrate (RuCl₃·xH₂O, CAS #14898-67-0) is purchased from ABCR chemical of which the product number is AB112073. This ruthenium precursor contains 38-43% in weight of Ru content. Commercial gamma-Al₂O₃ pellets were used as support material. The Al₂O₃ purity is confirmed with Xray Photoelectron Spectroscopy (XPS), which shows only characteristic peaks of Al, 0 and C due to surface carbonate adsorbents. The ammonia solution is prepared with a commercial ammonia aqueous solution (28-30 wt %) Sigma Aldrich (Product number #3587154) with deionized (DI) water.

### Step a) Pre-Treatment of Support Material

Commercial Al₂O₃ (shaped cylinder pellet) material (from AliExpress, cylinder shape with diameter 3 mm, length 4 mm with a surface area about 240 m²/g) is used as carrier/support of catalyst in the form of commercial Al₂O₃ pellets that were calcined at 500° C. to remove the water and adsorbents. For each batch of synthesis, the support material is first pretreated in an ammonia aqueous solution at 1-3M. The pellets are immersed in the solution for at least 3 to 15 hours and then washed with de-ionized (DI) water. The whole process can be done under atmospheric environment at room temperature. After washing with DI water, the pellets are then dried at 80-120° C. for about 2-15 hours. It has been checked that this pre-treatment does not change the porosity of the original Al₂O₃ support.

This steps not only enhances the CO₂ conversion performance but also improves the selectivity towards CH₄. A longer pre-treatment, typically from about 3 h to 15 h, leads to a higher low-temperature Ru mass specific reactivity of CH₄ yield.

### Step b) Preparation of Ruthenium Precursor Solution

Ruthenium Chloride hydrate (RuCl₃·xH₂O) or Ruthenium nitrosyl nitrate is used as precursor of Ru metal phase. An aqueous solution comprising less than 5 g/L of Ru ([RuCl₂(H₂O)₄]⁺+Cl⁻ in water) should be prepared freshly for each batch of synthesis while keeping it at low temperature (e.g. 3-20° C.) and avoiding long-term exposure to air. The concentration of Ru in the precursor solution is adjusted according to equation (1).

### Steps c) & d) Impregnation and Drying

Multi-steps incipient wetness impregnation is applied for the loading of ruthenium to the Al₂O₃ support. At each impregnation step an amount of the Ru in the precursor solution 1.2 times of the total volume of support is dropped onto the pretreated support, after the solution is filled into the pores of Al₂O₃ via capillary force, the impregnated product is dried at temperature between 20° C.-120° C. (e.g. 50° C., 80° C., 100° C. and 120° C.) for from 30 min to 2 h. A relation between the Ru size and evaporation rate of water has been clearly revealed. The higher evaporation rate leads to bigger Ru size as well as wider Ru size distribution due to the balance between the interaction and evaporation rate being disturbed. Therefore, drying with a lower evaporation rate (typically from about 0.2 mg/m²/min to about 1.2 mg/m²/min) is more favorable for obtaining small and homogeneous Ru size.

### Step e) Temperature Treatment & Reduction

After the impregnation and drying, the products are annealed under N₂-diluted H₂ gas environment with a concentration of H₂ between 1%-20% mol/mol (RuCl₃+H₂→Ru+HCl). The annealing temperature is between 300° C.-800° C., in particular from 400-800° C. (e.g. 400° C., 500° C., 600° C., 700° C. and 800° C.) with a heating rate less than 10° C./min and dwell time of at least 2 hours. The constant Ru size over different-temperature calcined Ru/Al₂O₃ catalysts can be ascribe to the strong adsorption between Ru precursor composites and Al₂O₃ as well as the highly dispersed Ru species during the previous steps.

### Step)) Catalyst Activation

After the annealing step, the obtained material is further immersed into 3M ammonia solution for at least 3 hours and then washed with DI water. This leads to the removal of residual Chloride in the Ru/Al₂O₃ catalyst: NH₄OH+HCl→NH₄Cl+H₂O and the sample after washing with ammonia solution exhibits a reactivity for CH₄ formation as tested in Example 7 which is 2 times more than that of the corresponding untreated catalyst. A conventional water washing of the catalyst shows a limited improvement compared to ammonia solution washing, indicating the chlorine is strongly attached into the catalyst. Therefore, the efficient removal of chlorine with a strong basic solution is necessary. Moreover, pretreatment with an ammonia solution before use will not only keep the basicity of catalyst but also avoid introducing other metal impurities (e.g., Na, K), which would harm the performance for CO₂ conversion and CH₄ selectivity. Therefore, step f) is preferably carried before the storage of the catalyst.

Activation step f) and drying step g) are conducted before storage in order to optimize the reactivity and stability of the catalyst product.

Although the residual of chlorine over supported Ru/Al₂O₃ catalysts were reported to have inhibition effect for H₂ adsorption during ammonia synthesis reaction (Miyazaki et al., 2001, Journal of Catalysis, 204, 364-37), some other studies show that chlorine existence can help activate the sites for H₂ adsorption (Lin et al., 2011, Catalysis Letters 141, 1557-1568; Gonzdlez-Carballo et al., 2015, Journal of Catalysis 332, 177-186). This contradictory discussion indicates that the impact of chlorine on the reactivity of Ru catalyst is still ambiguous, especially its influences on CO₂ methanation reaction are never investigated and reported.

Comparative sample: The comparative sample is prepared by conventional methods (Mi et al., 2012, Physics Procedia, 25, 1285-1291; US 20110014114; JP2008194615).

The commercial gamma-Al₂O₃ pellets with a surface area of 294 m²/g and total pore volume of 0.72 cm³/g was calcined at 500° C. to remove adsorbents. The Al₂O₃ pellets (no pre-treatment according to step a)) were then impregnated with an aqueous RuCl₃·xH₂O solution made with 0.0272 g of RuCl₃·xH₂O and 1.56 ml of DI water according to pore volume technique. The material was dried at 90° C. for 12 h in air and then annealed at 400° C. under 5% H₂, 95% N₂ gas environment for 2 hours. The reduced Ru/Al₂O₃ was then washed with DI water several times until no Cl⁻ was detected with AgNO₃ solution.

The % wt of Ru are determined by ICP-OES and the average ruthenium particle size by STEM analysis as described in the table below.

### Catalyst Characterization

The specific surface area, pore volume and average pore size of Al₂O₃ and Ru/Al₂O₃ samples were analyzed from N₂ adsorption-desorption isotherm using BELSORP MAX II (from MICROTRACT MRB). The specific area and the cumulative pores volume were obtained by the Brunauer-Emmett-Teller (BET) theory. The average pore size was obtained from the Barrett-Joyner-Halenda (BJH) model. Before the measurements, the samples were degassed under vacuum at 120° C. for 3 hours. using a BELP REP V AC II (from MICROTRACT). Xray photoelectron spectroscopy (XPS) of samples' wide scan from 1200 eV to 0 eV was measured by using the monochromated Kα line of an aluminum X-ray source (1486.6 eV) with the analyzer set at pass energy of 90 eV. The Agilent 5110 inductively coupled plasma optical emission spectrometry (ICP-OES) was used for checking the Ru content in final catalyst. The Ru particle size on Al₂O₃ support is analyzed based on the scanning transmission electron microscopy (STEM) images with ImageJ software, while the elemental distribution mapping is acquired by STEM energy dispersive X-ray (EDX). For each sample, the size distribution is based on measuring more than hundreds of particles with various microscopy images taken from different zones.

The evaporation rate of water in the pellet was measured with thermos-gravimetric analysis (TGA) was. The Al₂O₃ pellets were impregnated with certain amount of water. After impregnation, the pellets were left for 10 minutes before TGA was performed using a TG 209 F1 Libra by NETZSCH. The TGA was performed by increasing the temperature by 10° C./min until reaching the target temperature. The experiment continued isothermally until all the water evaporated. The target temperatures were set to 50° C., 80° C., 100° C., 120° C. and 150° C. The evaporation rate below 120° C. were calculated based on the TGA plot. For temperature more than 120° C., the evaporation rate of water is determined by model simulation.

### Example 1: Method of Preparation of Supported Ru Particles of the Invention

The method of the invention was applied to a porous Al₂O₃ support material with a BET surface area of 294 m²/g and a pore volume of 0.72 cm³/g.

### Step a) Pre-Treatment of Support Material

Two grams of commercial γ-Al₂O₃ pellets (AliExpress, cylinder shape with diameter 3 mm, length 4 mm) was immersed into 5 ml of 3M ammonia solution for 12 hours, and then dried at 90° C. for 12 hours.

### Step b) Preparation of Ruthenium Precursor Solution

0.0272 g of RuCl₃·xH₂O was dissolved in 3.256 ml of deionized water to have a Ru concentration of 5 g/L.

### Steps c) & d) Impregnation and Drying

The solution was then deposited on to the Al₂O₃ material by 2 impregnation steps. After each Ru solution impregnation, the Ru/Al₂O₃ sample was dried at 80° C. for 30 mins.

### Step e) Temperature Treatment & Reduction

The impregnated Ru/Al₂O₃ sample was then annealed in a tubular furnace at 400° C. for 2 hours in a gas mixture of 5% H₂ in N₂.

### Steps)) & g) Catalyst Activation

After annealing, the sample was immersed into 5 ml of 3M ammonia solution again for 3 hours and dried at 90° C. for 2 hours.

### Example 2: Method of Preparation of Supported Ru Particles of the Invention

The method of the invention was applied to a porous Al₂O₃ support material with a BET surface area of 242 m²/g and a pore volume of 0.80 cm³/g.

### Step a) Pre-Treatment of Support Material

2 grams of another commercial γ-Al₂O₃ pellets (Alfa Aesar) was immersed into 5 ml of 3M ammonia solution for 3 hours and then dried at 90° C. for 12 hours.

### Step b) Preparation of Ruthenium Precursor Solution

0.0272 g of RuCl₃·xH₂O was solved in 5.256 ml of deionized water to have a Ru concentration of 2 g/L.

### Steps c) & d) Impregnation and Drying

The solution was then deposited on to the Al₂O₃ material by 4 impregnation steps. After each Ru solution impregnation, the Ru/Al₂O₃ sample was dried at 80° C. in air for 30 mins.

### Step e) Temperature Treatment & Reduction

The impregnated Ru/Al₂O₃ sample was then annealed in a tubular furnace at 400° C. for 2 hours in a gas environment of 5% H₂ in N₂.

### Steps f) & g) Catalyst Activation

After reduction, the sample was immersed into 5 ml of 3M ammonia solution again for 3 hours and dried at 90° C. for 2 hours for stockage.

### Examples 3-6: Method of Preparation of Supported Ru Particles of the Invention

The Ru/Al₂O₃ catalyst is prepared by the same manner and using the same Al₂O₃ materials as in Example 1, except that the loading of Ru is different, corresponding to different times of impregnation as well as detailed in Table 1 below. In Example 6, The Ru/Al₂O₃ catalyst is prepared using the same material and process as Example 1, except that the temperature for reduction/annealing is 800° C.

### Example 7: Characterization of the Catalysts

The % wt of Ru and average size of Ru particles are compared for the various catalysts prepared by a method of the invention are compared with comparative commercial catalyst (CS) and comparative example (C) in Table 1 below.

TABLE 1

| | | | Average size | CO₂ conversion at |
|---|---|---|---|---|
| | Ru | Calcination | of ruthenium | p = 1 bar, |
| | loading | temperature | particle size | T = 285° C., |
| Sample | (wt %) | (° C.) | (nm) | GHSV = 6 L/g/h |
| CS | 0.47 | 400 | 1.83 ± 0.29 | 13.6% |
| C | 0.5 | 400 | 2.84 ± 1.13 | 32.15% |
| Ex. 1 | 0.45 | 400 | 0.90 ± 0.19 | 33.9% |
| Ex. 2 | 0.45 | 400 | 1.08 ± 0.19 | 34.6% |
| Ex. 3 | 0.8 | 400 | 0.94 ± 0.34 | 51.8% |
| Ex. 4 | 1.5 | 400 | 0.99 ± 0.21 | 63.5% |
| Ex. 5 | 2.5 | 400 | 1.39 ± 0.36 | 68.7% |
| Ex. 6 | 0.5 | 800 | 1.18 ± 0.24 | 34.6% |

As observed, by keeping the precursor solution as diluted not higher than 5 mg/ml of Ru, the average size of Ru is constantly kept under 1.5 nm for Examples 1 to 6 of the invention, while the Ru loading is increased up to 2.5%. The comparative catalytic evaluation over the treated Ru/Al₂O₃ catalyst and the conventional non-pretreated catalyst (in absence of step a) shows a dramatic difference of the CO₂ methanation reactivity, while the ammonia treated catalyst obtains much better CO₂ conversion under same condition.

This can be explained by the fact that the growth of Ru clusters on the support surface is well inhibited by the dispersed deposition of Ru species with a highly diluted precursor solution. The Ru species in the solution tends to be mounted on the surface of support separately after each impregnation until all the accessible adsorption sites get occupied by a small cluster. After all the active sites saturated with the small cluster of Ru species, more loading of Ru leads to growth of Ru clusters on local sites.

Ru-based egg-shell type catalyst is formed while using Al₂O₃ pellets (cylinder or spheres) as support by dry impregnation method. The thickness of the outer layer of Ru component obtained by a multi-step impregnation exhibited is about 400±37 m as measured by EDX-SEM. The penetrate of Ru precursor solution to the pellets center seems to be inhibited by stronger interaction between Ru composite and Al₂O₃ surface with a diluted ruthenium precursor solution. The strong interaction between Ru active and the adsorption sites on Al₂O₃ surface lead a stronger resistance towards agglomeration.

STEM-EDX elemental mapping results confirmed that the bright points are Ru particles, which are distributed homogenously onto the Al₂O₃ support for the materials of the invention which is not the case for the comparative materials where big agglomerated spots distributed randomly can be observed.

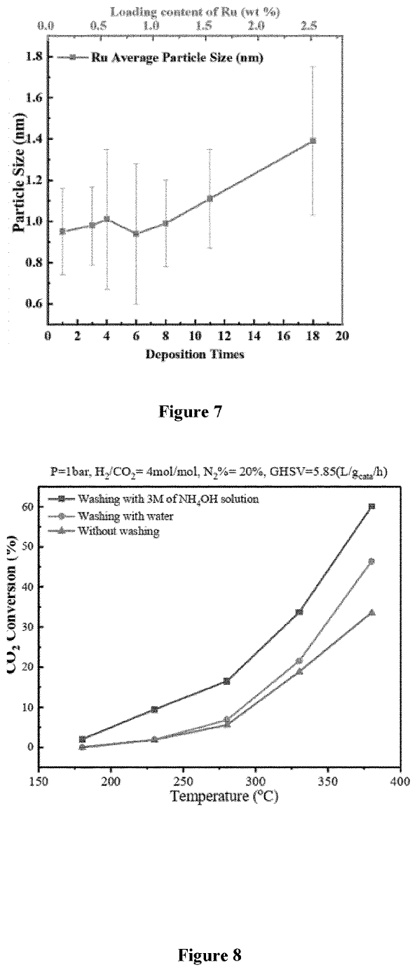

A study on the effect of the number of impregnation sequences revealed that if we keep the Ru precursor solution diluted (e.g. 3 mg/ml of Ru), the small Ru size of 1 nm can be maintained even with increasing the Ru loading from 0.5 to 1.5 wt %. In FIG. 7, it also demonstrates that while using a diluted RuCl₃·xH₂O solution (e.g. 3 mg/ml of Ru), the Ru particles size in a higher loading up to 1.5 wt % keeps below 1.5 nm even with 18 impregnation sequences.

The effect of the pre-treatment with an ammonia solution was tested as follows: a batch of Ru/Al₂O₃ catalysts with same loading (0.5 wt %) and same synthesis parameters as described above, except regarding the pre-treatment step a). They were divided to three batches with one of them were pretreated with hot water instead, while another was treated with 3M of NH₄OH solution according to the invention, respectively and another batch keeps unpretreated. The three batches were then tested for CO₂ methanation respectively under same conditions as described in Example 8 below. FIG. 8 shows the different CO₂ conversion of the three catalysts and demonstrates that the sample after washing with ammonia solution exhibits the highest reactivity for CH₄ formation, of which is 2 times more than that of the untreated catalyst. The conventional water washing method shows a limited improvement compared to ammonia solution washing, indicating the chlorine is strongly attached into the catalyst. Therefore, the efficient removal of chlorine with strong basic solution is essentially necessary. Moreover, pretreatment with ammonia solution will not only keep the basicity of catalyst but also avoid introducing other metal impurities (e.g., Na, K), which would harm the performance for CO₂ conversion and CH₄ selectivity.

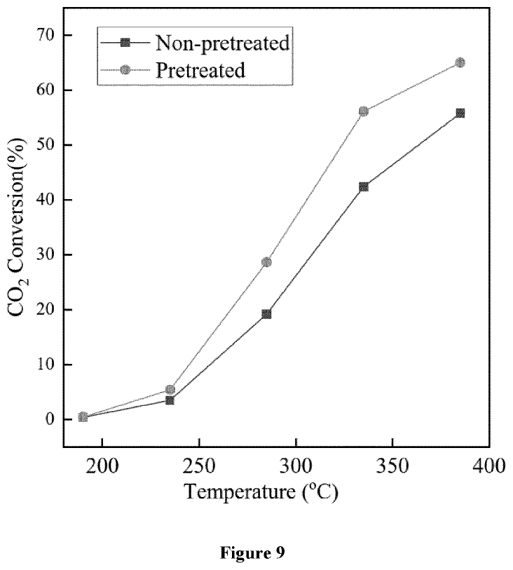

Another comparison was performed regarding CO₂ conversion between two Ru/Al₂O₃ samples synthesized using non-pretreated Al₂O₃ (Sigma Aldrich) and Al₂O₃ pretreated with ammonia solution (3M ammonia solution for 3 h), respectively. Throughout the synthesis, aside from the support pre-treatment, all other procedural steps remained identical, adhering to the specifications outlined in the method of the invention and employing consistent parameters. Ruthenium nitrosyl nitrate was used as the ruthenium precursor, the Ru loading was 0.5 wt %. The CO₂ reactivity tests were conducted under conditions detailed in the present application. The ensuing results are presented under FIG. 9 and unequivocally demonstrate the pronounced impact of the support pretreatment (step a) on the reactivity of CO₂ methanation.

### Example 8: Use of Supported Ru Particles of the Invention in CO₂ Methanation

The performance of the material of the invention used as a catalyst experiment in CO₂ methanation is assessed as described below.

200 mg of the materials described in the above Comparative Example, Example 1 and Example 2 were each loaded in a stainless-steel tube in a fixed bed flow reactor for CO₂ methanation reactivity test such as follows:

The reactor consisted of two 10 mm outside diameter sections of stainless tube, which serves as inlet and outlet and form a stainless-steel tube reactor of 8 mm inner diameter with 1 cm of length, in which the catalyst sample was placed by means of quartz wool. The reaction temperature was measured by a K-type thermocouple, which ran through the cell and contacted to the catalyst bed. The materials were heated in stream of 12 ml/min of diluted H₂ and at a rate of 10° C./min from room temperature to 400° C., which was maintained for 2 h and then cool down to 150° C. At 150° C., the reactants mixture stream of 22 ml/min of 18% of CO₂, 72% of H₂ and 10% of N₂ was replaced with a diluted H₂ stream. Based on the tests and Gas Chromatography (GC) quantification analysis, the CO₂ conversion and CH₄ selectivity can be obtained from the following equations:

$${CO}_{2} Conversion : {X}_{CO 2} = \frac{{CO}_{2 , in} - {CO}_{2 , out}}{{CO}_{2 , in}} \qquad ( 2 )$$

$${CH}_{4} Selectivity : {S}_{CH 4} = \frac{{CH}_{4 , out}}{{CO}_{, out} + {CH}_{4 , out}} \qquad ( 3 )$$

Whereas CO_{2, out/in}, CH_{4, out} are the molar concentrations at in/outlet of the reactor under each condition. The concentration of CO₂ and CH₄ is quantified by GC peak area multiplication with the response factor of each gas determined by external calibration. In principle, X_{CO2} and S_{CH4} is the straightforward data quantified by GC and used for the evaluation of catalysts' performance. Moreover, the injection and analysis of gas sample is obtained under a steady state condition, which refers to stable temperature, gas flow and pressure. N₂ is used as an internal standard reference gas to calibrate the concentration of reactants at the outlet of the reactor due to the total volume (pressure) drop. The mass specific CH₄ formation rate of ruthenium is determined by equation (4):

$${r}_{CH 4} = \frac{{X}_{CO 2} \times {S}_{CH 4} \times {F}_{CO 2}}{{m}_{Ru} \times {V}_{m}} \qquad ( 4 )$$

Wherein r_{CH4} is the formation rate of methane, unit is mol·g⁻¹_{Ru}·S⁻¹; X_{CO2} and S_{CH4} is CO₂ conversion and selectivity of CH₄ under a certain condition (pressure, temperature), respectively; F_{CO2} is the flow rate of CO₂ feed into the reactor, which is measured by mass flow controller, unit is L/S; m_{Ru} is the mass of Ruthenium used in each measurement, which is determined via weighting the catalyst loaded into the reactor multiply with the Ru weight content monitored by MS-ICP technique, unit is g; Vₘ is the molar volume of idealistic gas at standard condition, which is 22.4 L/mol.

The CO₂ conversion used for the calculation of CH₄ formation rate is taken from the kinetic region under constant measurement conditions (285° C., 1 bar, 6 L·g·h⁻¹) for all samples which reflects the intrinsic conversion of catalyst for comparison. The deviation for the CO₂ conversion is determined to ±2% and was determined by repeating the same measurement 3-5 times. FIG. 3 shows the reactivity of materials of Example 1 and 2 which exhibit higher mass specific CH₄ formation rate of Ru due to the better dispersion of ruthenium particle size. With the same amount of Ru mass, the catalyst synthesized via the process of the invention triggers at least 20% CH₄ formation compared to the comparative commercial catalyst (Sigma-Aldrich).

### Example 9: Stability of the Catalyst of the Invention

The stability and durability of a catalyst synthesis of the invention is assessed under harsh conditions of high pressure and temperature as described below.

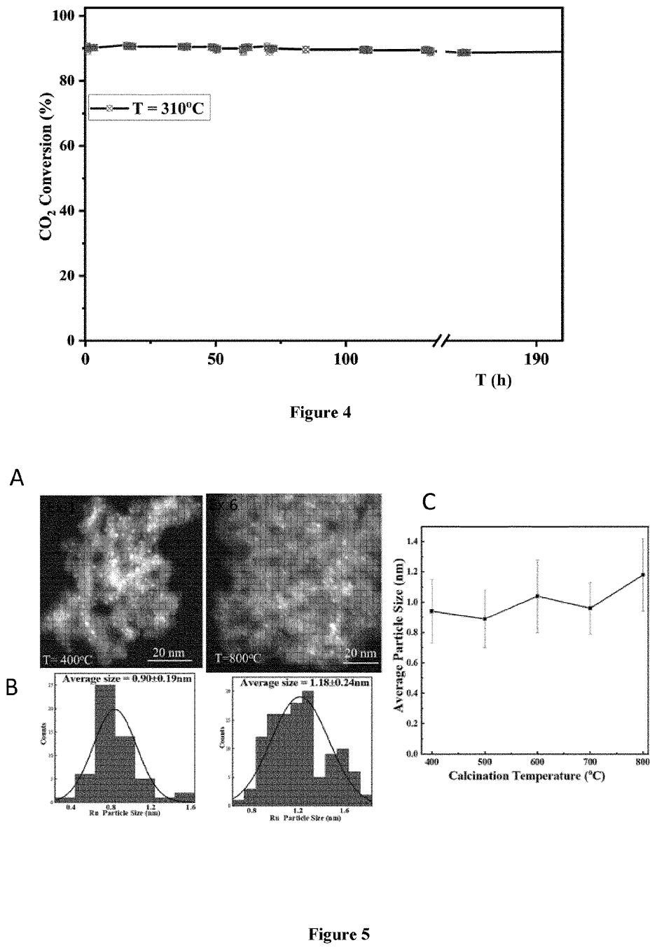

300 mg of the catalyst of Example 3 was loaded into a tubular fixed bed flow micro-reactor as described above. After reduction at 400° C. for 2 hours, 20% of CO₂ and 80% of H₂ were introduced into the reactor and heated up to 310° C. at 10 bar of total pressure in the reactor bed. The CO₂ conversion was continuously monitored by GC under this condition. The performance of the catalyst over time as measured by the % of CO₂ conversion as shown in FIG. 4 demonstrates that the CO₂ conversion keep constant up (as high as 97.8%) to 200 hours, confirming a high stability of Ru nanoparticles from the catalyst of the invention.

The STEM images of 50 h-spent catalyst show a homogenous distribution of Ru particles with an average size of 0.97 nm and that the same size and distribution of Ru particles are seen in the fresh catalyst.

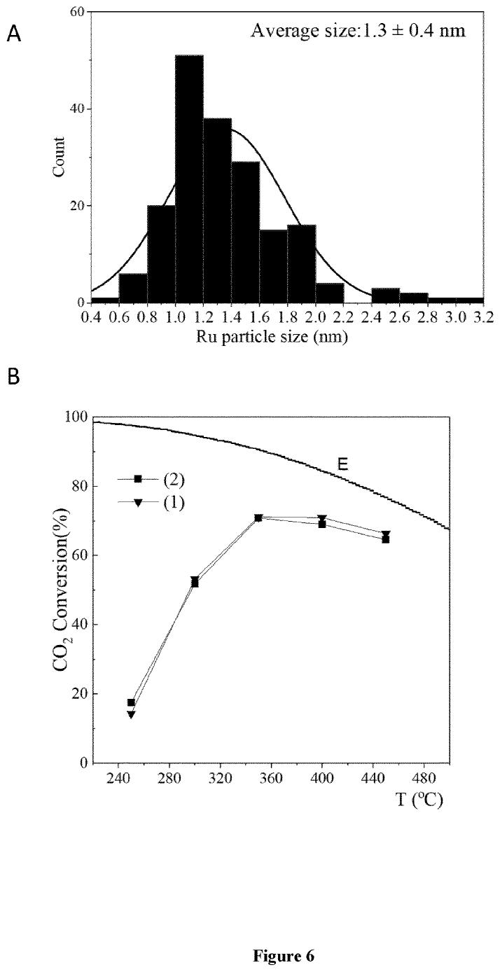

The stability of the catalyst of the invention is confirmed by the results from FIG. 5 which shows that the catalyst of Example 3 is resistant to agglomeration at high temperature up to 800° C., which has the great advantage to avoid a possible deactivation of catalyst due to hotspot in a methanation reactor, thereby impact the costs of production and the productivity of methanation units.

### Example 9: Scale Up of the Method of the Invention

The method of the invention was used to prepare 5 kg of a catalyst of the invention. About 5 kg of commercial Al₂O₃ pellets were pretreated in 15 L of 3M ammonia solution for 5 hours and then dried at 100° C. for 2 h. For each impregnation sequence, about 4 L of RuCl₃·xH₂O aqueous solution with a concentration of about 3 g/L was prepared and then impregned to the pretreated support material. Then, the Ru-impregnated Al₂O₃ was dried in oven under air at 100° C. for 2 hours. After 3 impregnation and drying sequences, the sample was annealed in 10 vol. % H₂ at 400° C. for 3 hours with heating rate of 1° C./min and gas flowrate of 1′000 L/h. After annealing and cooling, the sample was soaked in 3M of ammonia solution for 5 hours and then washed with cold water. After that, the sample was immediately dried overnight in air at 100° C. before storage.

As shown in FIG. 6A, the obtained catalyst has also highly dispersed and homogenous Ru particles with an average size of 1.3 nm. Furthermore, the catalytic activity assessed as described above demonstrates that the catalyst synthesized under industrial conditions has same CO₂ conversion as the catalyst synthesized in laboratory conditions via same the process of the invention (FIG. 6B) at P=1 bar, CO₂/H₂=4 mol/mol, N₂%=20%, GHSV=6 (L/g_{cat}/h).

Altogether, those data show that the method of the invention allows the preparation of Ru/Al₂O₃ catalyst with a mass loading of Ru from 0.1%_{wt} to 2.5%_{wt} with good methane yield for the CO₂ hydrogenation reaction (Sabatier reaction). Moreover, compared with commercial Ru/Al₂O₃ catalysts and catalysts synthesized via conventional methods, the catalysts of the invention present an active phase in which Ru metal is in a highly dispersed form which results in a higher mass specific reactivity of ruthenium compared to corresponding commercial catalysts and their production can be scaled up easily.

Furthermore, the catalysts of the invention are characterized by a strong interaction between the Ru nanoparticles and the Al₂O₃ support, can maintain a high degree of Ru metal dispersion without agglomeration during CO₂ methanation under harsh conditions.

1-20. (canceled)

21. A method for the preparation of Al₂O₃ supported Ru particles comprising the steps of:
a) Providing a porous Al₂O₃ support material which has been pre-treated in an ammonia solution;
b) Providing a ruthenium precursor solution comprising soluble ruthenium salts in aqueous solution, wherein the Ru mass content in the Ru precursor solution is from about 0.1 g/L to about 5 g/L;
c) Impregnating the surface of the said pre-treated Al₂O₃ support material with the ruthenium precursor solution by incipient wetness impregnation;
d) Drying the obtained impregnated Ru/Al₂O₃ material;
e) Subjecting the dried impregnated Ru/Al₂O₃ material to temperature treatment under an H₂ atmosphere to reduce the ruthenium precursor into Ru metal phase;
f) Contacting the obtained Ru/Al₂O₃ material to an ammonia solution; and
g) Drying the obtained ammonia-treated Ru/Al₂O₃ material.

22. The method according to claim 21, wherein the porous Al₂O₃ support material is provided in the form of cylindrically or spherically shaped Al₂O₃ pellets.

23. The method according to claim 21, wherein the porous Al₂O₃ support material is pre-treated in an ammonia solution with a concentration from 1M to 3M for from about 3 to about 15 hours.

24. The method according to claim 21, wherein the porous Al₂O₃ support material has a specific surface area over 100 m²/g and a total pore volume over 0.4 cm³/g.

25. The method according to claim 21, wherein ruthenium precursor solution comprising soluble ruthenium salts in aqueous solution, wherein the Ru mass content in the Ru precursor solution is about 3 g/L.

26. The method according to claim 21, wherein ruthenium precursor solution is a ruthenium chloride hydrate (RuCl₃xH₂O) solution.

27. The method according to claim 21, wherein ruthenium precursor solution is a ruthenium nitrosyl nitrate solution.

28. The method according to claim 21, wherein the pre-treated Al₂O₃ support material is subjected to a drying step for about 3-15 h prior the impregnation step.

29. The method according to claim 21, wherein the incipient wetness impregnation comprises at least one impregnation sequence of the following steps: i) wetting the pre-treated Al₂O₃ support material surface drop by drop with a volume of solution containing the ruthenium precursor corresponding to its pore volume until a slurry is finally formed when the whole pore volume of the support has been filled and ii) drying the impregnated Al₂O₃ support material by heating in air or vacuum conditions to about 50° C. to 100° C. for about 30 min to few hours.

30. The method according to claim 21, wherein at each impregnation sequence, after the Ru precursor aqueous solution is filled into the pores of Al₂O₃ via capillary force, the impregnated product is dried at temperatures between 20° C. and lower than 120° C. for about 30 min to about 2 h.

31. The method according to claim 21, wherein an evaporation rate of water less than 10 mg/m²/min is maintained during each drying step of the impregnation sequence.

32. The method according to claim 31, wherein the evaporation rate of water during each drying step of the impregnation sequence is between 0.2 mg/m²/min and 1.2 mg/m²/min (e.g. 0.5 mg/m²/min).

33. The method according to claim 21, wherein the temperature treatment of the dried impregnated Al₂O₃ support material is conducted in diluted H₂ atmosphere at a volume ratio from 1% to 20% in an inert gas of N₂, Argon or Helium.

34. The method according to claim 21, wherein the temperature treatment of the obtained dried impregnated Al₂O₃ support material is carried out at an annealing temperature from about 400° C. to about 800° C. with a heating rate less than 10° C./min and dwell time of at least 2 hours.

35. The method according to claim 21, wherein the annealed Ru/Al₂O₃ material is contacted with an ammonia solution.

36. The method according to claim 21, wherein the ammonia-treated annealed Ru/Al₂O₃ material is dried before storage.

37. A Ru/Al₂O₃ material obtainable by a method according to claim 21.

38. A heterogenous catalyst comprising Ru metal particles and Al₂O₃, wherein said catalyst has a mass loading of Ru from 0.1% wt to 2.5% wt and a size of Ru particles less than 2 nm.

39. The heterogenous catalyst according to claim 38, presenting a CO₂ conversion efficiency of more than 40% at a temperature from about to 250° C. to about 300° C.

40. A method for producing methane, said method comprising bringing a catalyst according to claim 38 into contact with a mixed gas containing CO and/or CO₂ and hydrogen gas at a temperature of at least 200° C.
